(19) United States
(12) Patent Application Publication
(10) Pub. No.: US 2023/0149830 A1
(43) Pub. Date: May 18, 2023

(54) Sonically Enhanced Microfiltration of Trichomes

(71) Applicant: David Profitt, Little Elm, TX (US)

(72) Inventor: David Profitt, Little Elm, TX (US)

(21) Appl. No.: 17/685,168

(22) Filed: Mar. 2, 2022

Related U.S. Application Data
(60) Provisional application No. 63/278,750, filed on Nov. 12, 2021.

(51) Int. Cl. B01D 11/02

(52) U.S. Cl. CPC B01D 11/0288; B01D 11/0284; B01D 11/0261

(57) ABSTRACT

A filtration system is described that can be used to filter out plant material within a solution. One or more sieves can be disposed in series to receive a flow of solution. As the solution flows through the one or more sieves, progressively smaller particles can be filtered out. One or more transducers can apply sound waves or other agitation during the filtering process. The sound waves help in suspending solid particles in the solution and making for more efficient and quicker filtering.

### CROSS REFERENCE TO RELATED INFORMATION

This application claims the benefit of U.S. Provisional Patent Application No. 63/278,750, filed Nov. 12, 2021, titled Sonically Enhanced Microfiltration of Trichomes, the contents of which are hereby incorporated herein in its entirety.

### TECHNICAL FIELD

The present disclosure is directed to filtration of plant trichomes.

### BACKGROUND OF THE INVENTION

Trichomes are fine outgrowths or appendages on plants, algae, lichens, and other plants. They are of diverse structure and function. Examples are hairs, glandular hairs, scales, and papillae. A covering of any kind of hair on a plant is an indumentum, and the surface bearing them is said to be pubescent. Trichomes sometimes have medicinal or nutritional value. The collection and filtering of trichomes from other plant parts can be time consuming and expensive.

### BRIEF SUMMARY OF THE INVENTION

One embodiment under the present disclosure comprises a filtration system. The system comprises a plate configured to receive filtration media thereon and to allow a flow of fluid to pass therethrough. It can further comprise a frame coupled to the plate and configured to hold the plate above a receptacle; and one or more transducers coupled to the plate and configured to agitate the plate during a filtration process.

One embodiment under the present disclosure comprises a filtration system. The filtration system comprises one or more sieves configured to receive a flow of solution therethrough, the solution comprising a fluid and a material. The system can further comprise a receptacle configured to receive the flow of solution as it exits the one or more sieves; and a fluid reservoir configured to store the solution. Further, it can comprise a supply line coupled to the fluid reservoir and configured to provide the flow of solution to the one or more sieves; a return line coupled to the reservoir and the receptacle and configured to recycle filtered solution to the reservoir; and one or more transducers configured to agitate the one or more sieves during the flow of solution.

A further embodiment comprises a method of filtering under the present disclosure. The method can comprise directing a flow of solution through one or more sieves, the solution comprising a fluid and a plant material. Further steps include agitating the one or more sieves as the solution flows through the one or more sieves and collecting the filtered plant material collected by each of the one or more sieves.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

For a more complete understanding of the present invention, reference is now made to the following descriptions taken in conjunction with the accompanying drawings, in which:

FIG. 1 shows a diagram of a system embodiment under the present disclosure;

FIG. 2 shows drum and sieve embodiment under the present disclosure;

FIG. 3 shows a digital signal processor embodiment under the present disclosure;

FIG. 4 shows a diagram of a system embodiment under the present disclosure;

FIG. 5 shows a diagram of a system embodiment under the present disclosure;

FIG. 6 shows an embodiment of a transducer under the present disclosure; and

FIG. 7 shows a flow-chart of a method embodiment under the present disclosure.

### DETAILED DESCRIPTION OF THE INVENTION

Trichomes from plants can be useful for medicinal uses, food and other uses. But the medicinal or food content can be difficult to isolate from other plant materials. In one example of the prior art, plant material is frozen and dropped in an ice bath. The material is then heated, resulting a murky soup that can then be put through multiple levels of filtration. This can result in filtered trichome glands containing the desired plant material. In some cases the plant soup is filtered through multiple drums of filters and water. The drums, filter, water, and other materials can be heavy to manipulate, requiring multiple people for a relatively low output of material. This process can be time consuming, labor intensive, and consume a lot of resources.

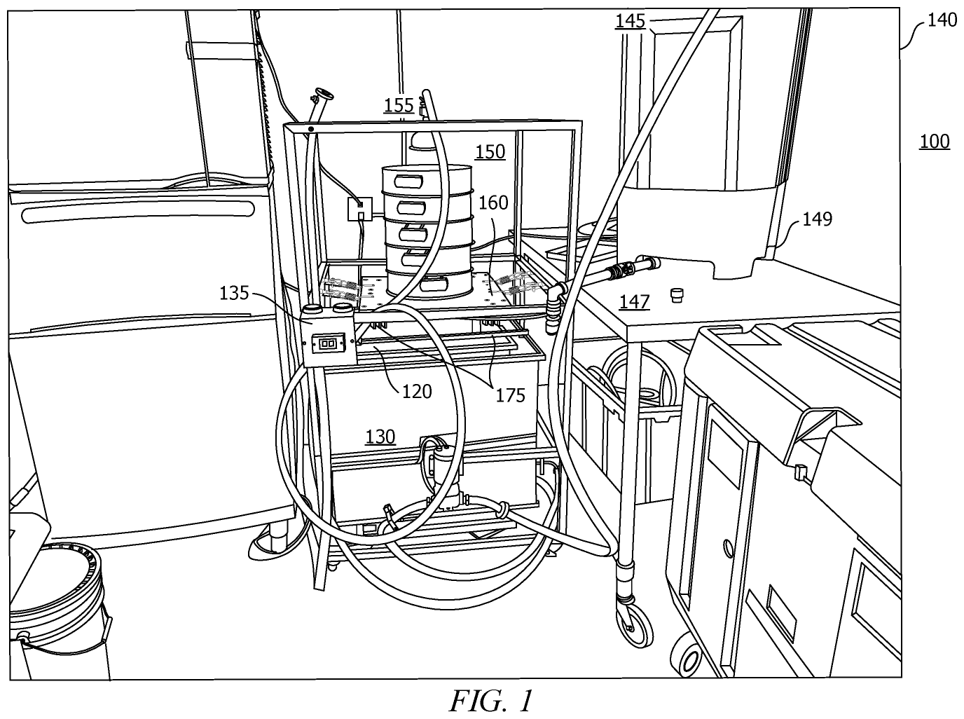

Embodiments under the present disclosure include improved filtration devices and processes. One embodiment of a filtration system 100 is shown in FIG. 1. Drum or sieve stack 150 sits on plate 160 which can be suspended over, or sit on top of, drainage receptacle 130. Power supply 135 can turn on power to the system and connect to or comprise an electrical power supply via battery, wall connect, or other means. Reservoir 140 can hold water or another fluid. Supply line 145 connects to the top of drum 150 and can provide a flow of fluid over plant material within drum 150 via outlet 155. Within drum 150 are one or more levels of filtration media (not shown in this figure) of decreasing size going down the drum 150. At each level of filtration a finer size of plant material or trichomes will be filtered out of the plant material and fluid. Return line 147 can return used fluid to the reservoir 140. Drainage receptacle 130 can contain or be coupled to a cleaning system to clean the fluid before it returns to reservoir 140. Pump 130 can couple to the supply 145 and return lines 147 and pump fluid to and from reservoir 140. Drainage receptacle 130 preferably contains an inner receptacle 120. Inner receptacle 120 receives the flow-through from the bottom of drum 150. Drainage receptacle 130, sitting around inner receptacle 120, preferably contains ice to help keep the fluid and other components cold. Wash basin 149 forms a portion of reservoir 140 and can receive plant material and/or recycled fluid from drum 150 and drainage receptacle 130.

One or more transducers 175 are coupled to plate 160. As fluid pours from outlet 155 over drum 150 and flows through the plant material, the transducers 175 can be turned on. Applying agitation, vibration, oscillation, and/or sounds waves from transducers 175 helps to keep the particles in the liquid in suspension such that plant material doesn't rest on screen/filter heads in the filtration media. This allows the water to flow through plant material and sieves of the filtration media more quickly than a standard filtration system without transducers 175. Whereas the prior art could take an hour to filter a batch of plant material, embodiments under the present disclosure can filter the same size batch in 7-15 minutes. Higher quality filtering, with better filtering of trichome sizes, can be achieved as well.

Transducers 175 preferably are capable of agitating the plate 160 and thereby agitating drum 150 and filtration media. The agitation can produce sound waves or produce vibration or oscillation in a variety of frequencies. Certain embodiments of the present disclosure are effective at filtering cannabinoids from cannabis trichomes. In such embodiments, using frequencies from generally 25-2500 Hertz has been found to be effective. Other embodiments may use other frequencies, however. Effective frequencies may vary depending on plant material type or amount, temperature, fluid or solution composition, and other factors. It may be desirable to vary frequencies during filtration to help provide a thorough filtration. The agitation emitted by transducers 175 are preferably physical waves that are not necessarily in the hearing range of human beings. In many embodiments these waves may not be audible to human beings or may sound like a low rumble.

After filtering, the transducers 175 can be turned off and the flow of fluid turned off. Trichomes, or other desired filtered material, can be harvested from each level of drum 150. For cannabis and THC (tetrahydrocannabinol), a typical filtration session can cause trichomes of roughly 175-180 microns to filter out at one level of filtration media, down to roughly 40 microns at a bottom level. Different size trichomes, or different grades, may be more or less valuable and may have different preferred uses in the cannabis industry. Embodiments described herein can be applicable to a variety of different plant and/or filtering applications. While some reference is made to cannabis, other embodiments are possible. For different plants, or different filtered material, different frequencies may yield different results, and different size filtration media may be used while keeping with the teachings described herein.

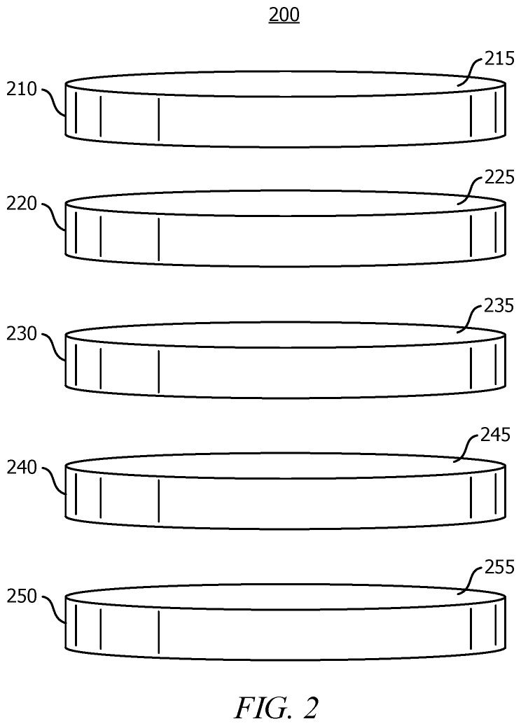

FIG. 2 shows an exploded view of a drum 200. Levels 210, 220, 230, 240, 250 can each comprise filtration media 215, 225, 235, 245, 255. The filtration media preferably becomes finer going down the stack. Stainless steel sieves, for example, can be used to collect valuable material at different micron layers as fluid passes through levels 210-250. Levels 210-250 can be integrated together or can comprise individual stackable layers. Filtration media 215-255 can comprise sieves, meshes, screens and/or other filter types operable to filter any fluid, plant material, or other filtered material. Drum 200 is preferably open on the bottom so as to allow fluid to flow to a receptacle.

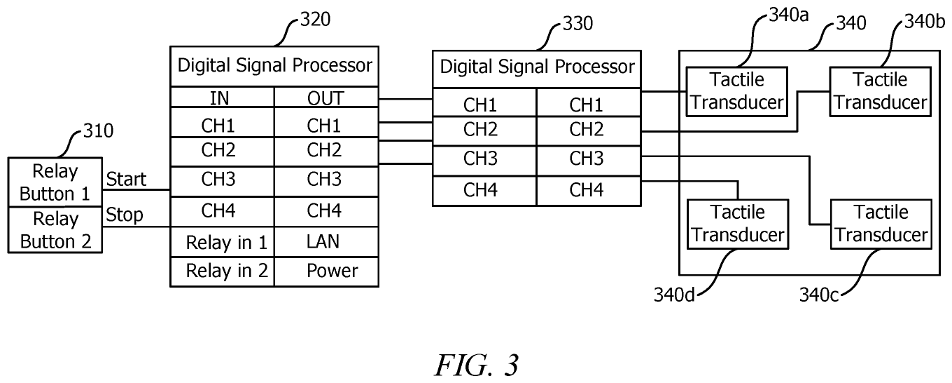

FIG. 3 shows a possible embodiment of an electrical diagram of a system under the present disclosure. Relay 310 can be communicatively coupled to digital signal processor 320, which can be communicatively coupled to amplifier 330, which can be communicatively coupled to the tactile transducers 340a-d on transducer plate 340. Other embodiments can comprise other quantities of tactile transducers, channels, amplifiers, relays, power supplies, and other components. The digital audio signal processor 320 can be programmed to create and distribute a sound wave intermittently to four tactile transducers 340a-d in turn. By being intermittent, this audio signal distribution method can help in keeping the transducers cool and operating for as long as possible. An intermittent signal creates less strain and gives a longer service life to transducers 340a-d and other components. The sounds wave vibrations on the drum can be introduced through the transducers 340a-d, themselves mounted to a transducer plate either on top or below depending upon the environment. The transducer plate preferably has a large opening meant to mount and hold the sieves for collection. As the vibrations pass throughout the sieve stack, the materials collected on the screens can remain in suspension within the fluid so as to allow the water/fluid to pass through the sieve stack in an expedited manner.

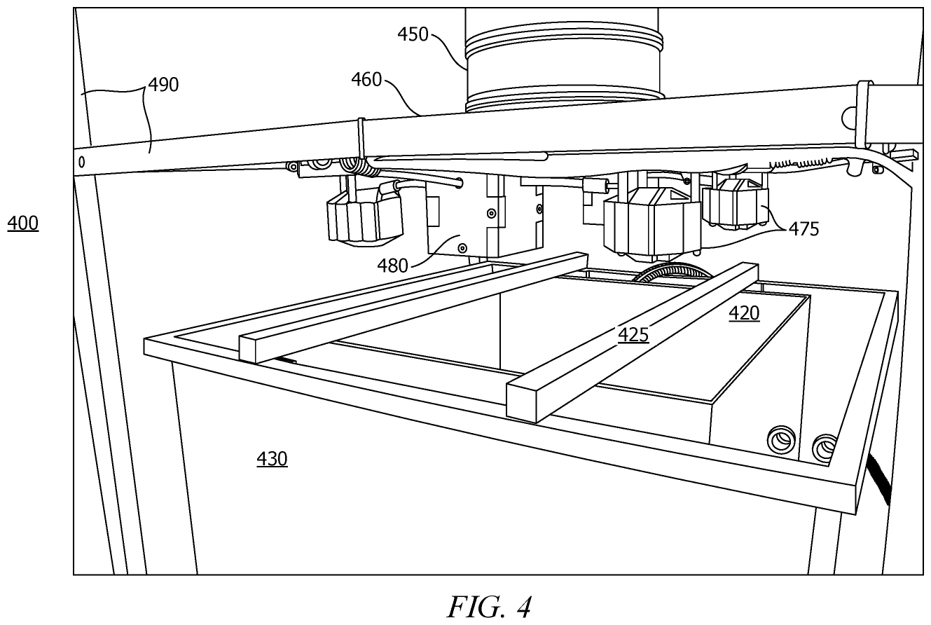

FIG. 4 shows another view of a filtration system embodiment. Drum 450 sits on plate 460 which is coupled to framing 490. Drum 450 is positioned about inner receptacle 420 and outer receptacle 430. Bars 425 can sit on inner receptacle 420 or outer receptacle 430 to help prevent drum 450 or plate 460 from falling down. Transducers 475 can be coupled to plate 460, or framing 490, or another component. Computing device 480 can comprise a relay, digital signal processor, amplifier, power supply or connection to power supply or other components. Outer receptacle 430 preferably surrounds inner receptacle 420 such that ice can be filled into outer receptacle 430 to keep the contents of inner receptacle 420 cold.

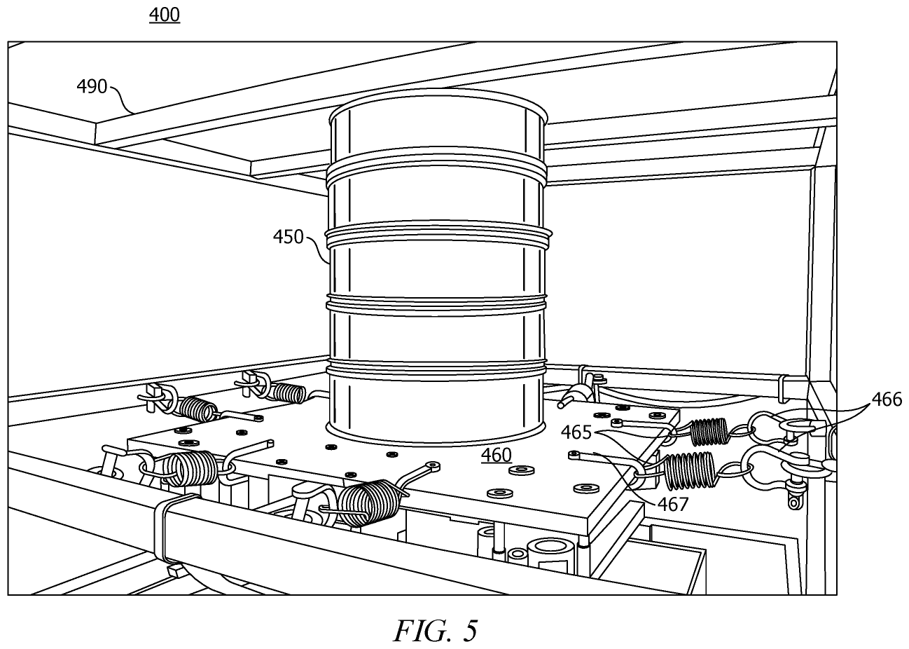

FIG. 5 shows another view of the filtration system 400 of FIG. 4. Drum 450 with sieves inside sits on plate 460. Frame 490 provides supporting structure, in this case resembling a cube. Springs 465 can couple plate 460 to frame 490. Springs 465 can allow the drum 450 and plate 460 to vibrate in response to sound waves from transducers 475 (see FIG. 4). Other coupling means are possible between the plate 460 and frame 490. Depending on the type of springs 465 or coupling used, the type of transducers 475 or frequencies used may vary. As seen in FIG. 5, springs 465 can be coupled to the frame 490 via a bolt connection 466. Clamps 467 can couple the springs 465 to plate 460. Other connection means are possible. Bolts, screws, clamps, wraps, adhesives, welding, sauntering, and other means are all possible.

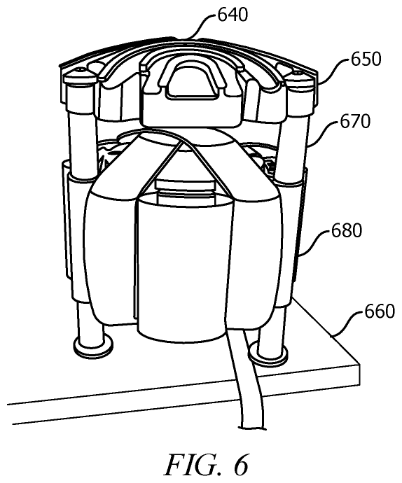

FIG. 6 shows an embodiment of a tactile transducer 650 coupled to a plate 660. Bolts 670 can couple transducer 650 to plate 660. Other coupling means, such as sauntering, welding, bolts, screws, washers, other means, and combinations of the foregoing, are all possible. Cap 640 provides protection and bolting connections while still allowing transducer 650 to provide agitation to the filtration system. Housing 680 can house a piston and circuitry for controlling and adjusting the transducer 650. Transducer 650 is shown here coupled to the plate 650. However, coupling could be done anywhere that a tactile transducer such as transducer 650 can agitate filtration media within the filtration system. Transducers as shown in FIG. 6 and other embodiments can be prone to overheating. Many transducer embodiments comprise a piston as a portion of the sound creation means. The physical movement of the piston can add to the creation of heat. Some piston embodiments also help to increase the flow of air through the transducer, helping to alleviate some of the heat. The amount of heat created may vary depending on transducer and piston embodiments.

To alleviate and avoid overheating, it is preferred to rotate or alternate which transducer is activated. For example, transducers can be configured to apply sound in various stages. In an embodiment comprising four transducers, one, two, or three transducers can apply agitation while the other transducer(s) rests. The pattern of applying agitation could be two transducers on for five minutes, while the other two are off. After five minutes the transducers can be reversed, and so forth. In embodiments comprising two transducers, only one transducer might be activated at a time. Any desired number, or proportion, of available transducers can be chosen to run at the same time. One preferred embodiment comprises four transducers and a filtration process wherein one transducer is activated at a time while the other three rest. The activated transducer is rotated after a chosen length of time, possibly five minutes. The length of operation may depend on the transducer type or size, settings or factors within the filtration system such as load or power limitations, or environmental factors such as temperature or humidity.

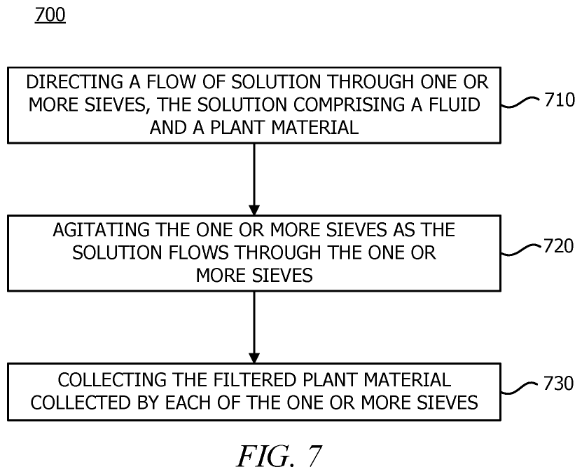

FIG. 7 shows a possible method embodiment under the present disclosure. Method 600 is a method of filtering plant material. Step 710 is directing a flow of solution through one or more sieves, the solution comprising a fluid and a plant material. Step 720 is agitating the one or more sieves as the solution flows through the one or more sieves. Step 730 (optional, or carried out separately, in some cases) is collecting the filtered plant material collected by each of the one or more sieves. The agitation under this method can be provided by one or more transducers coupled to, or near, the one or more sieves. The one or more transducers can be activated in a rotating pattern so as not to overheat any individual transducer. Furthermore, optionally, the method of FIG. 7 can comprise steps for creating the solution. Creating the solution can include: freezing the plant material; placing the frozen plant material in an ice bath; and agitating the solution to separate the plant material.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

What is claimed is:

1. A filtration system comprising:
a plate configured to receive filtration media thereon and to allow a flow of fluid to pass therethrough;
a frame coupled to the plate and configured to hold the plate above a receptacle; and
one or more transducers coupled to the plate and configured to agitate the plate during a filtration process.

2. The filtration system of claim 1 further comprising a controller configured to activate the one or more transducers during filtration processes, the controller comprising a digital signal processor configured to transmit a digital signal for each of the one or more transducers.

3. The filtration system of claim 1 further comprising one or more springs configured to couple the plate to the frame.

4. The filtration system of claim 1 wherein each of the one or more transducers comprises a piston.

5. The filtration system of claim 2 wherein the controller is configured to alternate which of the one or transducers to activate during a filtration process.

6. The filtration system of claim 1 further comprising one or more sieves comprising filtration media and configured to couple to the plate to and allow the flow of fluid therethrough.

7. The filtration system of claim 6 wherein the one or more sieves are configured such that each sieve is operable to filter progressively smaller particles as fluid flows through the one or more sieves.

8. The filtration system of claim 1 further comprising a receptacle configured to collect fluid flowing through the plate.

9. A filtration system comprising:
one or more sieves configured to receive a flow of solution therethrough, the solution comprising a fluid and a material;
a receptacle configured to receive the flow of solution as it exits the one or more sieves;
a fluid reservoir configured to store the solution;
a supply line coupled to the fluid reservoir and configured to provide the flow of solution to the one or more sieves;
a return line coupled to the reservoir and the receptacle and configured to recycle filtered solution to the reservoir; and
one or more transducers configured to agitate the one or more sieves during the flow of solution.

10. The filtration system of claim 9 wherein a first of the one or more sieves is configured to filter particles sized 175-220 microns.

11. The filtration system of claim 9 wherein a final of the one or more sieves is configured to filter particles sized 35-40 microns.

12. The filtration system of claim 9 wherein the solution comprises water and cannabis.

13. The filtration system of claim 12 wherein the one or more sieves is configured to filter out tetrahydrocannabinol trichome glands.

14. The filtration system of claim 9 wherein each of the one or more transducers comprises one or more pistons.

15. The filtration system of claim 9 wherein the one or more transducers are configured to provide agitation in the range of 25-2500 Hertz.

16. The filtration system of claim 9 wherein the one or more transducers are configured to vary the frequency of the agitation during the filtering process.

17. A method of filtering, comprising:
directing a flow of solution through one or more sieves, the solution comprising a fluid and a plant material;
agitating the one or more sieves as the solution flows through the one or more sieves; and
collecting the filtered plant material collected by each of the one or more sieves.

18. The method of claim 17 further comprising creating the solution by;
freezing the plant material;
placing the frozen plant material in an ice bath; and
agitating the solution to separate the plant material.

19. The method of claim 17 wherein the agitating is performed by a plurality of transducers coupled to a plate coupled to the one or more sieves.

20. The method of claim 19, wherein during a first time period a first of the plurality of transducers performs the agitating and during a second time period a second of the plurality of transducers performs the agitating.
